(19) United States
(12) Patent Application Publication
(10) Pub. No.: US 2003/0026728 A1
(43) Pub. Date: Feb. 6, 2003

(54) Substance and microclimate simulator

(76) Inventor: Vlad Iorgulescu Avram, Toronto (CA)

Correspondence Address: Peter Milne, Gowling Lafleur Henderson LLP, Suite 4900, Commerce Court West, Toronto, ON, M5L 1J3, CA

(21) Appl. No.: 09/920,990

(22) Filed: Aug. 3, 2001

(51) Int. Cl. A61L 9/00

(52) U.S. Cl. 422/4; 422/123; 422/124; 422/125; 422/5; 422/305; 422/306

(57) ABSTRACT

A substance distribution apparatus has a receiver for receiving a signal containing at least a substance component. The apparatus further has a substance generator for producing a plurality of desired substances. A processor communicates with the receiver and the substance generator to interpret the desired substance signal and causes the substance generator to generate and release selected of the desired substances which correspond to the desired substance signal. The apparatus may further include tactile features such as a water mist generator and temperature regulation.

### FIELD OF THE INVENTION

This invention relates generally to entertainment devices. More particularly this invention relates to substance generation in a multi-media presentation, special effects including climate simulation, containment and evacuation of contaminated mediums in indoor or outdoor spaces (if more than one unit is used) and crowd control.

### BACKGROUND OF THE INVENTION

Traditional theatres appeal to an audience's sense of sight and hearing. The performance, whether a motion picture or theatrical includes a choreographed performance using sets or props and a combination of dialogue and music.

The audience however has five senses: sight; sound; smell; touch; and taste. Accordingly, while theatrical or motion picture presentations set out to create an illusion of reality for an audience, the result is achieved limited by the fact that at least half of the sensations that would normally accompany certain sights and sounds are missing.

For example, a real-life thunderstorm not only carries flashes of lightening, claps of thunder and the sound and sight of rain falling, it is also accompanied by the substance of ozone and wet earth, a change in temperature and a change in humidity.

As further examples, a drive through a coniferous forest would in the real world carry the substance of pine and a walk along the seashore would include a cold breeze and the substance and taste of salt sea air. A walk through a garden presents the substance of flowers and a pub generally reeks of cigarette smoke and beer.

It is an object of this invention to provide means to appeal at least to the sense of smell and, to a more limited extent, the tactile senses of a motion picture or theatre audience in conjunction with an audio-visual presentation.

It is a further object of the present invention to provide apparatus which may be used for crowd control purposes and evacuation and containment of contaminated mediums in indoor or outdoor spaces.

### SUMMARY OF THE INVENTION

A substance generating apparatus is provided which has a receiver for receiving a signal (manual or encoded) containing at least a substance component. The apparatus includes a substance generator for dispensing a plurality of desired substances. A processor communicates with the receiver and the substance generator for interpreting the signal and causing the substance generator to generate and release selected of the desired substances which correspond to the signal.

The signal may be stored along with a visual image on an image storing media. The image storing media may be film, magnetic tape or a laser disc such as commonly referred to a as a CD or a DVD. The signal may be manually generated such as with an electronic keyboard or a switching device.

The substance generator may include at least one substance emitter for releasing at least one member selected from the group consisting of aerosol releasable substances and thermally releasable substances.

The apparatus may be a “stand alone” unit or may include connecting means for connecting the apparatus to a heating ventilating and conditioning system of a theatre to produce fluid communication between the released substances and the system thereby utilizing the system to carry the released substances into the theatre.

A plurality of units may be used particularly where containment and evacuation is desired.

The signal may include a temperature component and the processor maybe connected to the heating, ventilating and air conditioning system. The processor may interpret the temperature component and direct an appropriate response in the system.

The signal may further include a humidity component and the generator may include a water mist producer. The processor may interpret the humidity component and cause an appropriate response in the water mist producer.

The substance emitter may include a plenum, a plurality of substance storage and release means operably connected to the processor for releasing substances into the plenum, a pressurized air source for providing air to the plenum and an outlet for discharging substance laden air from the plenum.

The substance storage and release means may include pressurized aerosol containers.

The substance storage and release means may be a magazine having a plurality of substance cartridges mounted about a spindle rotatable by a drive means communicating with the processor. A pressurized air source may be selectably connectable to the cartridges to provide fluid communication for ejection of substances from the cartridges by pressurized air from the pressurized air source.

The substance storage means may be a thermally releasable medium on a substance scroll. The scroll maybe passed over a substance release means in the form of a heater within the plenum. The processor may be operably connected to the heater and to a scroll drive means for winding the scroll to pass the scroll over the heater to cause the heater to heat the thermally releasable medium to release selected of the stored substance into the plenum.

A method is provided for containment and evacuation of a contaminated medium, the method includes the steps of:

(i) operating a fan in an evacuation mode for drawing air away from an affected area;

(ii) directing air jets at the affected area to direct contaminated air to the fan;

(iii) capturing contaminated air with the fan;

(iv) separating the contaminated medium from the air and storing the contaminant.

The method may be modified by introducing a neutralizing or other substance (including liquids) into the air jets in step (ii) contaminated area.

### DESCRIPTION OF DRAWINGS

Preferred embodiments of the present invention are set out below with reference to the accompanying illustrations in which:

FIG. 1 is a schematic representation of a substance substance distribution apparatus according to the present invention;

FIG. 2 is a partially cut away pictorial representation of a substance emitter according to the present invention for releasing aerosol carried substances;

FIG. 3 is a perspective view of a substance magazine;

FIG. 4 is a section or line 4-4 of FIG. 3 but also illustrating substance release apparatus and a processor for use with the substance magazine of FIG. 3;

FIG. 5 is a partially cut away pictorial representation of a substance emitter according to one embodiment of the present invention for releasing thermally releasable substances;

FIG. 6 is a schematic representation of a stand alone unit according to the present invention; and,

FIG. 7 is a further schematic representation of a stand alone unit according to the present invention.

### DESCRIPTION OF PREFERRED EMBODIMENTS

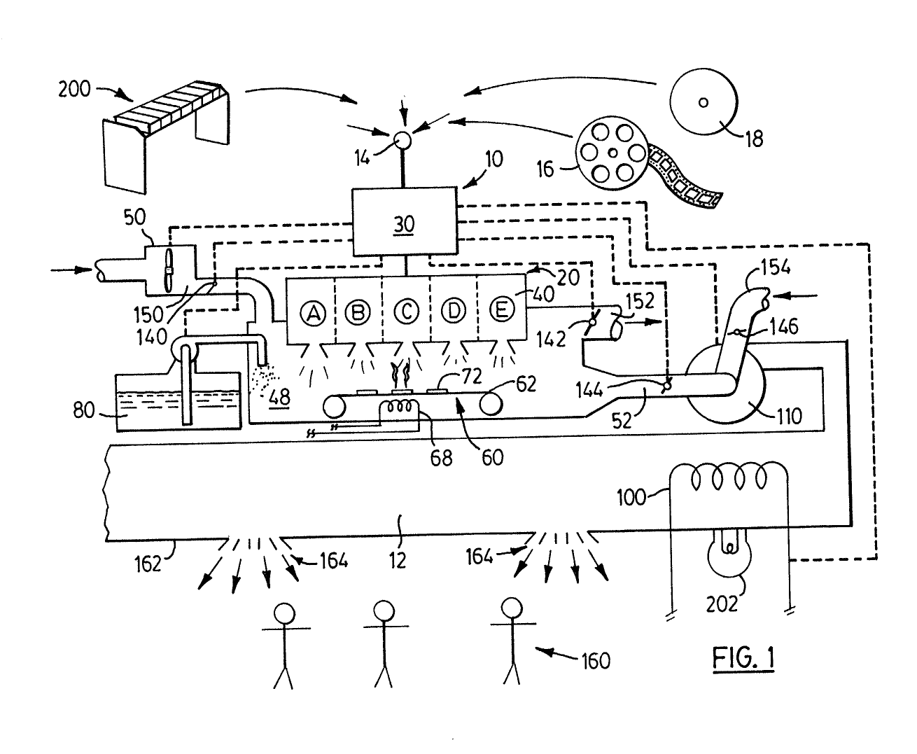

A substance substance distribution apparatus according to the present invention is generally indicated by reference 10 in FIG. 1. The apparatus 10 in FIG. 1 is illustrated as being connected to a heating ventilating and air conditioning (“HVAC”) system 12 such as might be found in a theatre or other building structure. While this is not essential, it is a beneficial way of taking advantage of building's air distribution system to carry substances and tactile stimuli. The device could however have its own delivery system, which would be a requirement where such a system is not present, such as an open air situation.

The apparatus 10 has a receiver 14 for receiving a signal from a source such as a magnetic tape or film sound track 16 or a laser disc (“CD” or “DVD”) 18. The signal might be ancillary to other information stored on the storage media such as video or audio information, somewhat like the practice of having a sound track carried by a motion picture filmstrip or a video tape.

Alternatively, the signal might be manually generatable such as with an electronic keyboard 200 or other switching device.

The signal would contain information on at least a desired substance. It may include information on a plurality of substances or additional information such as temperature, humidity, windspeed etc. Alternatively, particularly in cases of keyboard-like signal generators, substance information and additional information may be independently provided using different keys or other switches.

The apparatus 10 has a substance generator which is generally indicated by reference 20. The substance generator 20 is capable of producing a variety of substances in a manner which is described in more detail below. A processor 30 communicates with the receiver to interpret (decode) the signal and to cause the substance generator to release the desired substance. For example, the substance information may relate to flowers and be stored along with a video image of a walk through a flower garden. The processor will receive the signal from the storage madia, and cause the release of a floral fragrance to coincide with the display of the video image.

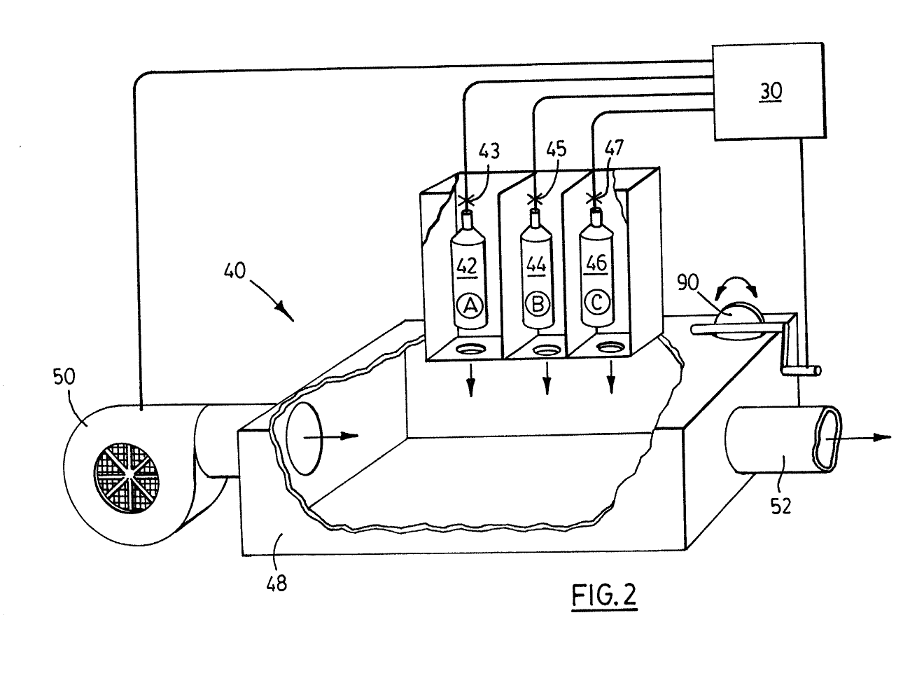

The substance generator 20 may include one or more substance emitters such as emitter 40 for releasing aerosol carried substances and emitter 60 for releasing thermally released substances. Representative substance emitters are illustrated in FIGS. 2 through 5 and described in more detail below. Any type or number of substance emitters may be connected to the substance generator 20. Some of the emitters may carry “stock” substances which are commonly utilized. Other emitters may carry custom substances more specific to a particular presentation. Preferably any storage and release means for custom substances will provide ease of access and exchangeability.

Other apparatus for producing tactile stimulus may also be incorporated in the substance generator 20. For example a water mist producer (atomizer) 80 may be utilized to create damp or foggy conditions. Furthermore the substance generator 20 itself or associated HVAC equipment may have a heater such as shown at reference 100 which is also controlled by the processor 30. The processor may also control output of a fan 110 associated with the HVAC system 12 or separate therefrom to control airflow around an audience 160, for example to simulate wind.

FIG. 2 illustrates a substance emitter 40 for releasing aerosol carried substances. These may include such substances as ozone (electrical storm), hydrogen sulphide (rotten eggs), or other gasses. The aerosol substances may also include essential oils and possibly powders.

The substance emitter 40 has a series of pressurized canisters 42,44 and 46, each containing a desired substance (substance storage means). The canisters fluidly communicate with a plenum 48 into which the aerosol canisters 42,44, and 46 may be selectively released upon a signal from the processor, for example by the triggering of respective solenoid valves 43, 45 and 47 (substance release means).

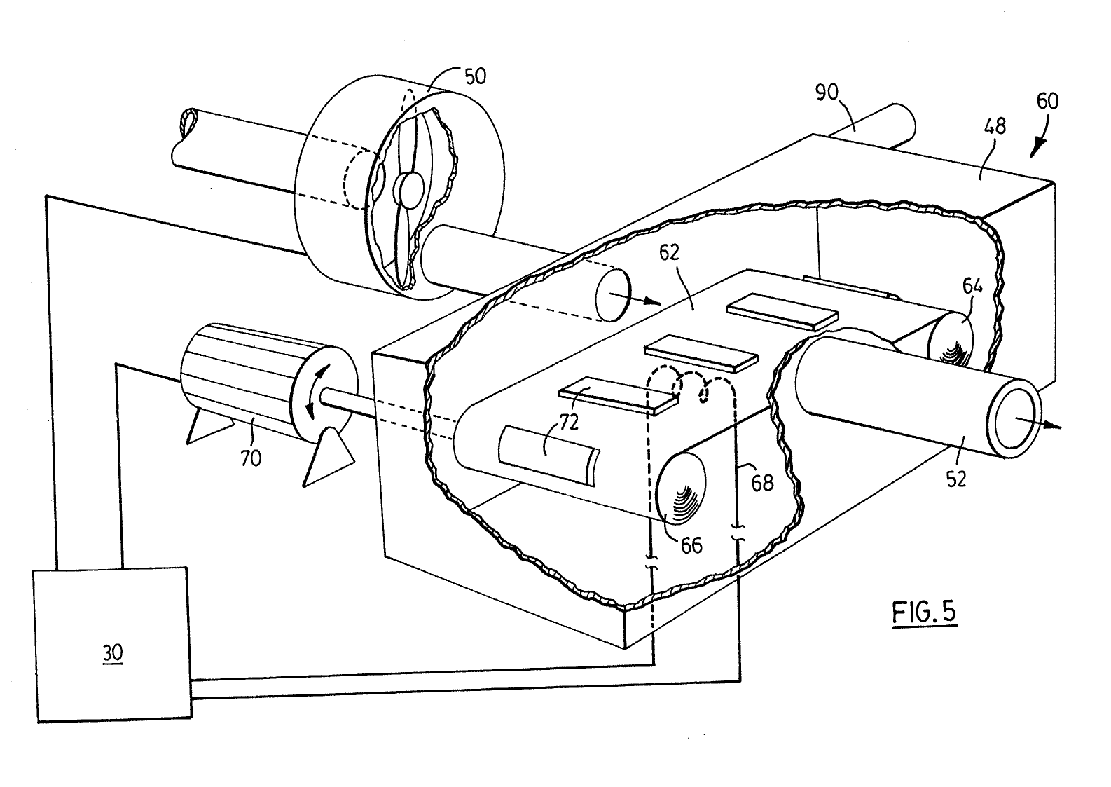

A fan 50 may be provided to carry the released substances (fragrances) from the plenum 48 through an outlet 52. The fan 50 and plenum 48 may be specific to one type of substance emitter, as shown in FIGS. 2 and 5, or may be common to different types of emitters such as emitters 40 and 60 in FIG. 1.

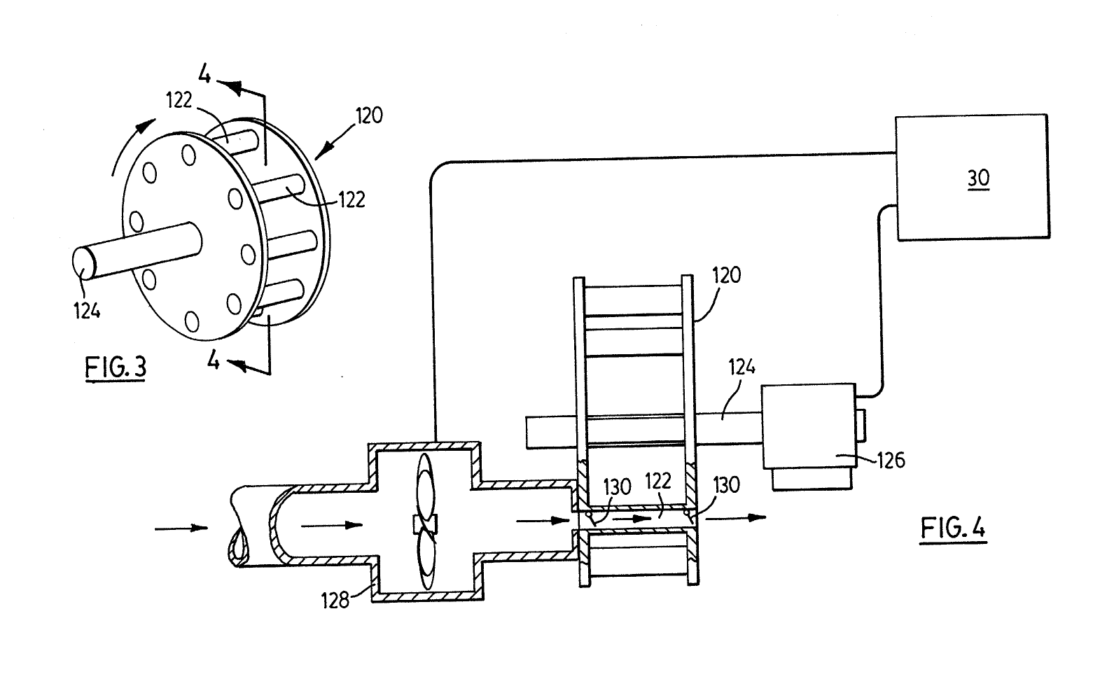

FIGS. 3 and 4 illustrate a substance storage means in the form of a magazine 120 which is a replaceable/exchangeable component that carries a plurality of substances in individual chambers or cartridges 122. The chambers 122 may be oriented about a spindle which may in turn be rotated by suitable drive means such as a motor 126. Each chamber may be pressurized, or alternatively be movable in front of a pressurized air source such as a fan or compressor 128. The controller 30 communicates with the compressor 128 (substance release means) and motor 126 to coordinate release of a selected substance. Release may be effected by blowing open pressure valves 130 at opposite ends of the chamber 120 to eject the substance.

The use of a substance magazine 120 enables replacement of spent substance cartridges 122 or exchange of substance cartridges 122 for ones with different substances with relative ease, which may for example be effected between performances. The substance magazine 120 also makes possible the supply of substance arrays to accompany the supply of an audio or audio visual storage media (such as a VCR tape or DVD).

Another substance emitter is illustrated by reference 60 in FIGS. 1 and 5. The substance emitter 60 contains a scroll 62 which is wound from a first spool 64 onto a second spool 66 over a heater 68 (substance release means). A motor 70 or other drive means may be provided to wind the scroll. The scroll 62 has on its surface a plurality of thermally releasable substance strips 72 (substance storage means).

The scroll 62 and heater 68 may be mounted within a separate plenum or within the main plenum 48 as shown in FIG. 1.

A separate fan or the system fan 50 maybe used to carry released substances from the plenum 48 through the outlet 52. The processor 30 may control variables such as scroll movement, heater timing, fan force and fan timing to coordinate substance release with visual or other media being presented.

In order to avoid confusing substances or causing inappropriate substance combinations, provision may be made to purge the “stand alone” emitters such as emitters 40 and 60 illustrated in FIGS. 2 and 5 respectively, such as by providing a purge outlet 90 directed away from an audience 160. In the combined system 20 of FIG. 1, this may be accomplished by providing servo-controlled valves 140, 142, 144 and 146 respectively in an inlet 150 to the plenum 48, in an exhaust outlet 152 from the plenum 48, between the plenum 48 and an inlet to the HVAC fan 110 and a fresh air inlet 154 to the HVAC fan 110.

Closing the valve 144 and opening the valves 142 and 140 will allow the fan 50 to purge the plenum 48 through the exhaust outlet 152, which preferably exhausts away from an audience 160, and out of the building if the apparatus is mounted in such.

Closing the valve 144 and opening the valve 146 allows the fan 110 to draw in fresh air from an inlet 154 and direct it through ductwork 162 and out of vents 164 to clear the air surrounding the audience 160. The valves 140, 142, 144 and 146 may be controlled by the processor 30.

The above description basically relates to a substance substance distribution apparatus principally for use in association with motion picture entertainment. Other applications are however possible. For example, the signal may be produced by other apparatuses such as a keyboard 200 which may be manually actuated to release desired substances upon selection of an appropriate key or set of keys by an operator. Furthermore, non-substance related stimuli may be produced such as light displays using an associated light generator (laser, strobe etc.) 202 associated with the apparatus. The light display may for example be a laser picture show and might accompany a musical presentation.

In addition to an entertainment function, the apparatus of the present invention may be utilized to dispense other air or gas born substances. For example, if crowd control is required in an area served by the apparatus 10, unpleasant substances (hydrogen sulphide, skunk etc.) or even crowd control gases (tear gas) may be dispensed by one of the substance emitters such as emitter 40 or emitter 60 as appropriate depending on the properties of the substance to be dispensed.

The device 10 might even find application in situations where protection may be required from toxic substances such as might be dispersed in a crowd through terrorist activity. The substance to be dispensed by the emitter may include neutralizing substances effective against various biological agents that could be dispensed if biological threat is perceived.

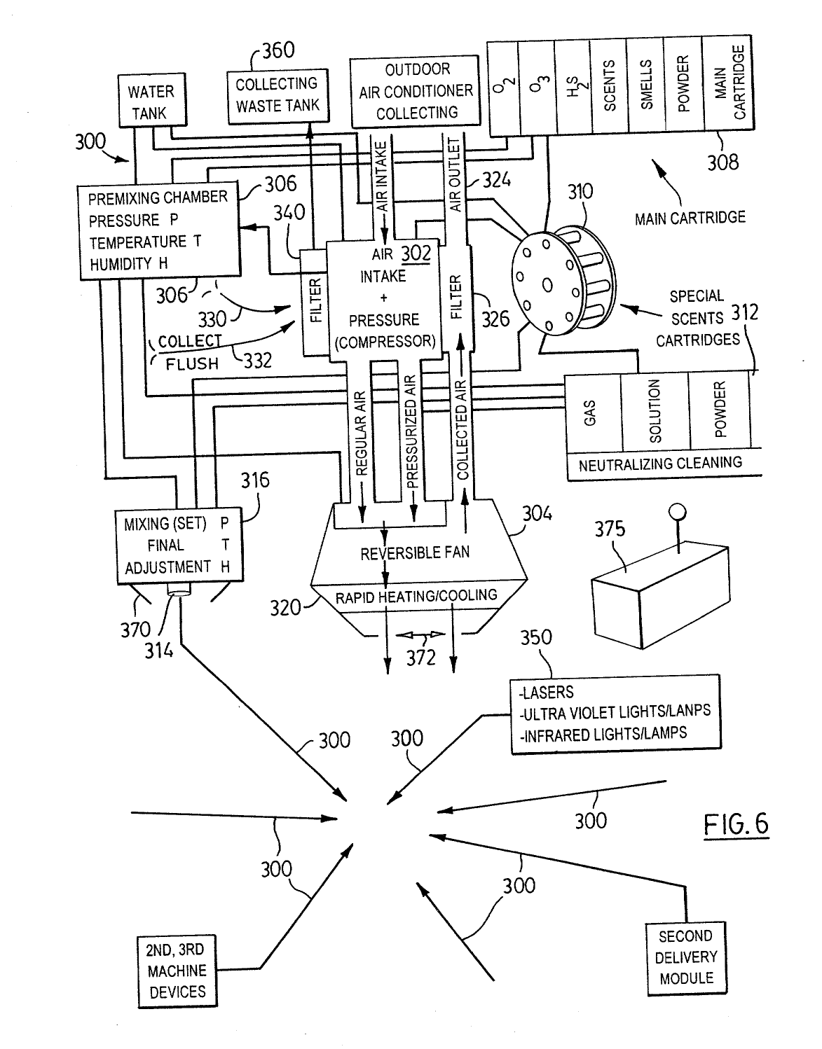

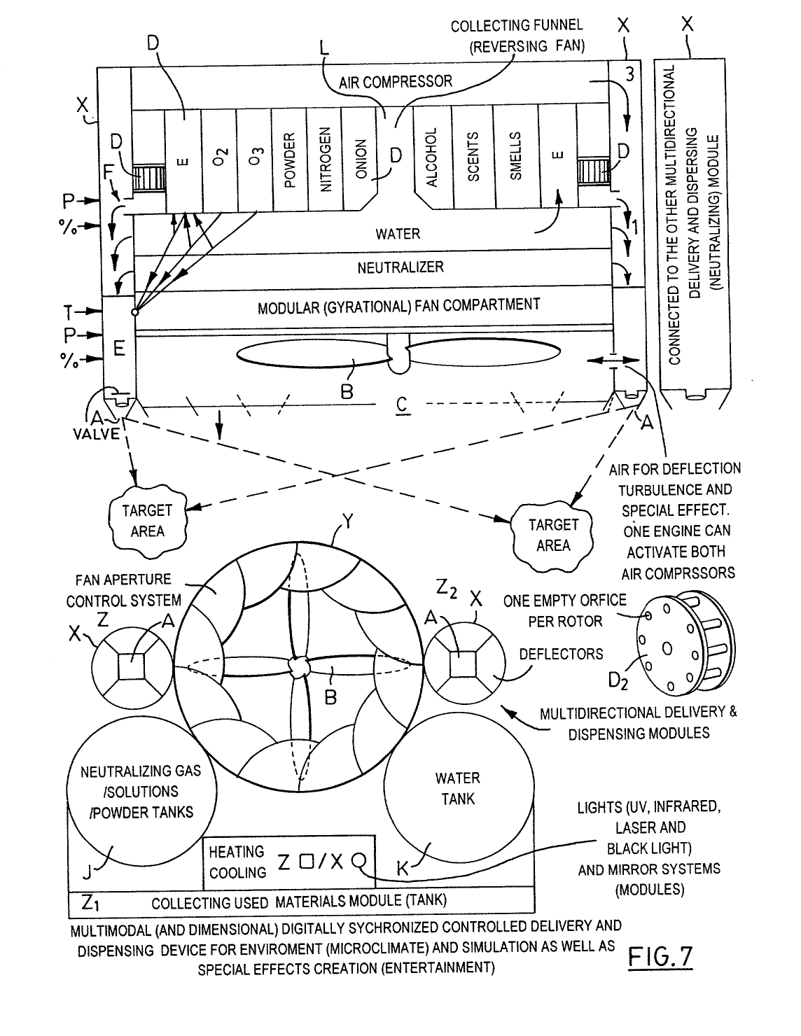

FIG. 6 illustrates a “stand alone” apparatus 300 according to the present invention. The apparatus 300, rather than relying on an existing air delivery air system (such as a building's HVAC system) has its own air delivery system. The air delivery system includes both a compressor 302 for generating pressurized air, principally for use in short bursts or high velocity directed streams and a fan 304 for general delivery. The compressor 302 may pressurize a premixing chamber 306 which communicates with substance (or other substance) emitters 308, 310 and 312. The premixing chamber may in turn communicate either with directional jets 314 and a final mixing chamber 316 or with the fan 304. A heat exchanger 320 maybe provided in association with the fan 304 for rapid cooling and heating. The substance emitters 308, 310 and 312 may communicate directly with the fan 304.

The fan 304 maybe of a reversing nature to either blow air into or withdraw air from an area adjacent thereto. Substances (or other airborne substances) which are withdrawn may be discharged through an outtake 324 and may initially be filtered through a filter 326 if it is desired to remove substances from the collected air. The air compressor 302 may be used to flush the premixing chamber 306 and the final mixing chamber 316 as desired through respective collector outlets 330 and 332 which in turn may be processed through a filter 340 and discharged or stored.

A plurality of devices 300 may be utilized, particularly for a large venue as a rock concert or an open air venue. The plurality of devices 300 may be used in conjunction, for example with one reversing fan 304 drawing air away from the venue (acting as a “captivating and exhausting” fan) and the remaining fans 304 and directional jets 314 acting as containment and directing streams for causing air from the venue to be directed toward the captivating and exhausting fan. The units 300 can be used with any combination of lasers, ultra violet light, infrared light, strobe lights and other special effect apparatus as generally indicated by reference 350.

It will be appreciated that having a plurality of stand alone units may be quite effective if it becomes necessary to contain and remove infected or contaminated air from a venue. For example each of the units 300 may be provided with neutralizing cartridges (for example as part of the emitter 310) containing antiseptics or antitoxins which may discharged into a contaminated venue and the contaminated air directed by the directional jets 314 to the containment and exhaust fan 304. The contaminated air can be treated or collected as appropriate, for example a waste tank 360 may be provided.

The directional jets 314 have the added benefit of being able to create high velocities and turbulence for added special effects. Adjustable deflectors or diffusors 370 and 372 may be provided respectively in association with the jets 314 and the fan 304. As the FIG. 1 illustration, a processor 375 communicates with the balance of the system and receives appropriate encoded input which it processes and in turn controls system parameters such as pressures, temperatures, velocities, direction, special effects, fan direction and substance discharge.

The above description is intended in an illustrative rather than a restrictive sense. Variations maybe apparent to those skilled in such apparatus without departing from the spirit and scope of the present invention as defined by the claims set out below.

I claim:

1. A substance distribution and generation apparatus comprising:
a receiver for receiving at least a signal containing a substance component;
a substance generator for dispensing a plurality of desired substances;
a processor communicating with said receiver and said substance generator for interpreting said signal and causing said substance generator to release selected of said desired substances which correspond to said signal.

2. The substance distribution and generation apparatus of claim 1 wherein:
said signal is stored along with a visual image or an image storage media.

3. The substance distribution and generation apparatus of claim 2 wherein:
said image storage media is selected from the group consisting of film, magnetic tape and a laser disc (“CD or DVD”).

4. The substance distribution and generation apparatus of claim 3 wherein:
said substance generator includes at least one substance emitter for releasing at least one member selected from the group consisting of aerosol releasable substances and thermally releasable substances.

5. The substance decoding an generating apparatus of claim 4 wherein:
said apparatus includes connecting means for connecting said apparatus to a heating ventilation and air conditioning system of a theatre to provide fluid communication between said released substances and said system thereby utilizing said system to carry said released substances into said theatre.

6. The substance distribution and generation apparatus of claim 5 wherein:
said signal further includes a temperature component;
said processor is connected to said heating ventilating and air conditioning system;
said processor interprets said temperature component and directs an appropriate response in said system.

7. The substance distribution and generation apparatus of claim 6 wherein:
said signal includes a humidity component;
said apparatus includes a water mist producer;
said processor is connected to said water mist producer, interprets said humidity component and causes an appropriate response in said water mist producer.

8. The substance distribution and generation apparatus of claim 4 wherein said substance emitter includes;
a plenum;
a plurality of substance storage and release means operably connected to said processor for releasing said substances into said plenum;
a pressurized air source for providing air to said plenum; and,
an outlet for discharging substance laden air from said plenum.

9. The substance distribution and generation apparatus of claim 7 wherein said substance emitter includes;
a plenum;
a plurality of substance storage and release means operably connected to said processor for releasing said substances into said plenum;
a pressurized air source for providing air to said plenum; and,
an outlet for discharging substance laden air from said plenum.

10. The substance distribution and generation apparatus of claim 8 wherein:
said substance storage and release means are stored in pressurized aerosol containers with remotely actuatable release valves.

11. The substance distribution and generation apparatus of claim 8 wherein:
said substance storage and release means is a magazine having a plurality of substance cartridges mounted about a spindle rotatable by a drive means communicating with and controlled by said processor;
a pressurized air source is selectably connectable to said cartridges to provide fluid communication for ejection of said substances from said cartridges by pressurized air from said pressurized air source.

12. The substance distribution and generation apparatus of claim 8 wherein:
said substance storage and release means is a thermally releasable medium on a substance scroll;
said substance scroll is passable over a substance release means in the form of a heater within said plenum;
said processor is operably connected to said heater and to a scroll drive means for moving sections of said scroll over said heater to cause said heater to heat said scroll and said thermally releasable medium on said sections of said substance scroll to release selected of said substance sources into said plenum.

13. The substance distribution and generation apparatus of claim 1 wherein:
said signal is produced by an operator using a manually actuatable apparatus.

14. A substance distribution and generation apparatus as claimed in claim 13 wherein:
said manually actuatable apparatus is a keyboard.

15. The substance distribution and generation apparatus of claim 14 wherein:
said substance generator includes at least one substance emitter for releasing at least one member selected from the group consisting of aerosol releasable substances and thermally releasable substances.

16. The substance distribution and generation apparatus of claim 15 wherein:
said apparatus includes light generating means responsive to said keyboard.

17. The substance distribution and generation apparatus of claim 1 wherein:
said desired substances included at least one member selected from the group consisting of crowd control substances, antibacterial substances, antiviral substances, antitoxins and antivenoms.

18. A method is provided for containment and evacuation of a contaminated medium, the method includes the steps of:
(i) operating a fan in an evacuation mode for drawing air and substance away from an affected area;
(ii) directing/substance air jets at the affected area to direct contaminated air to the fan;
(iii) capturing contaminated air with the fan; and,
(iv) separating the contaminated medium from the air and storing the contaminant.

19. The method of claim 18 including the further step of:
introducing a neutralizing substance to said contaminated air.

20. The method of claim 19 wherein said fan and said air jets form a part of separate and discrete distribution and generation apparatus comprising:
a receiver for receiving at least a signal containing a substance component;
a substance generator for dispensing a plurality of desired substances;
a processor communicating with said receiver and said substance generator for interpreting said signal and causing said substance generator to release selected of said desired substances which correspond to said signal.
